(19) United States
(12) Patent Application Publication
(10) Pub. No.: US 2019/0358308 A1
(43) Pub. Date: Nov. 28, 2019

(54) THROMBOXANE RECEPTOR-BASED VACCINE FOR MANAGING THROMBOGENESIS

(71) Applicant: Board of Regents, The University of Texas System, Austin, TX (US)

(72) Inventors: Fadi T. Khasawneh, El Paso, TX (US); Fatima Z. Alshbool, El Paso, TX (US); Zubair A. Karim, El Paso, TX (US)

(21) Appl. No.: 16/374,390

(22) Filed: Apr. 3, 2019

Related U.S. Application Data
(60) Provisional application No. 62/652,518, filed on Apr. 4, 2018.

(51) Int. Cl. A61K 39/00; A61K 9/00; A61K 39/39

(52) U.S. Cl. CPC A61K 39/0005; A61K 9/0019; A61K 39/39; A61K 2039/545; A61K 2039/55511; A61K 2039/58; A61K 2039/54; A61K 2039/55505

(57) ABSTRACT

A vaccine against TPR's ligand binding domain, namely the C-terminus of the second extracellular loop (C-EL2), inhibits platelet activation and thrombus formation, without exerting any effects on hemostasis.

### CROSS-REFERENCE TO RELATED APPLICATIONS

This application is related to and claims the benefit of priority of provisional U.S. Patent Application Ser. No. 62/652,518, filed Apr. 4, 2018, which is hereby incorporated by reference.

### BACKGROUND OF THE INVENTION

### A. Field of the Invention

The invention generally concerns the field of medicine. In particular the modulation of platelet aggregation.

### B. Description of Related Art

Upon injury to a blood vessel, the subendothelial matrix that is normally shielded from platelets gets exposed revealing many agonists and factors that are critical for platelet adherence and subsequent activation. Consequently, platelets will interact with the exposed subendothelial matrix, leading to intraplatelet signaling and activation, which is associated with a number of events, including release of arachidonic acid (AA) from the membrane phospholipids. The liberated AA is metabolized by the cyclooxygenase enzyme (COX-1) and thromboxane A₂ synthase, leading to the formation of thromboxane A₂ (TXA₂).

To this end, TXA₂, a well-studied platelet agonist, is a labile lipid mediator that acts through binding to its G-protein coupled receptor, namely the thromboxane A₂ receptor (TPR). This interaction causes a wide variety of biological effects, including platelet aggregation. To this end, TPRs' clear role in normal hemostasis is supported by the finding that “patients” have a bleeding disorder as a result of a point mutation in the receptor protein. On the other hand, upregulated signaling through TPR has been implicated in the pathogenesis of multiple cardiovascular and thrombosis-based diseases.

Consistent with this concept are clinical findings indicating that inhibition of platelet TXA₂ production provides therapy for thromboembolic diseases; which is the underlying rationale for the use of aspirin in such diseases. Consequently, the TXA₂ pathway has been targeted for pharmacological intervention either to inhibit its formation or to modulate binding to its receptor. In light of this fact, several possibilities have emerged to achieve this goal with COX inhibitors and thromboxane synthase (TS) inhibitors being the initial lead candidates.

The COX inhibitor aspirin remains the only clinically approved agent for therapeutic interventions that target the TXA₂ pathway. However, even though the aspirin is in clinical use, it is associated with many inherent limitations, including sever adverse effects (e.g., bleeding), resistance, amongst others. For example, the aspirin was found to: (1) lack selectivity to TXA₂ as it also inhibits PGI₂ synthesis, (2) cause bleeding and gastric ulcers—adverse effects that in some instances mandate its discontinuation, (3) redirect AA metabolism to isoprostanes, which themselves modulate platelet function, and (4) be associated with sensitivity and an increasing rate of resistance worldwide.

Thus, there is a need for other therapeutic agents for modulation of thromboembolic diseases.

### SUMMARY OF THE INVENTION

The inventors describe a vaccine to induce an antagonistic TPR response that can be used to modulate thromboembolic conditions. It became clear that a more selective means of blocking TXA₂-mediated platelet aggregation needed to be developed, and the inventors contemplated a receptor blockade as a promising approach.

Based on these considerations, the inventors sought to further assess the contributions of the C-EL2 domain to in vivo TPR-dependent platelet activation (e.g., hemostasis/bleeding time) and the genesis of thrombosis, by employing a vaccine-based approach employing the cognate TPR C-EL2 peptide as an immunogen. To this end, immunization of mice with the CEL2 peptide TPR vaccine did indeed lead to the production of a C-EL2 TPR antibody, and inhibit aggregation induced by the TPR agonist U46619, whereas it produced no effects on aggregation stimulated by separate agonists, namely ADP and TRAP4. Moreover, platelets from the immunized mice also exhibited selective defects in TPR-dependent dense and alpha granule release, and GPIIb-IIIa. In terms of its in vivo activity, the TPR C-EL2 vaccinated mice exhibited a prolonged time for occlusion, but their bleeding time was no different from the controls. On the other hand, vaccinating mice with the random version of the C-EL2 peptide (i.e., C-EL2r Vac) or KLH exerted no effects on platelet function, in vitro and in vivo.

Together, these findings indicate that the TPR C-EL2-based vaccine protects against thrombogenesis, without impairing hemostasis. Amongst other advantages, this active vaccination approach would not be expected to face some of the functional limitations an antagonist does, including frequent administration and high costs.

Embodiments generally provide compositions that induce antibodies that bind thromboxane (TX) A₂ receptors (TPR) and inhibit thrombosis and other events within the cardiovascular, renal or pulmonary systems. Compositions of the invention prevent TXA₂ from binding to the TPR and stimulating platelet activation and aggregation, thereby decreasing the risk of a clinically significant thrombus or embolus. Thus, the C-EL2 vaccine provides beneficial pharmaceutical properties for treating thrombosis and other events within the cardiovascular, renal or pulmonary systems.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or any variation of these terms includes any measurable decrease or complete inhibition to achieve a desired result.

The term “effective,” as that term is used in the specification and/or claims, means adequate to accomplish a desired, expected, or intended result.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 is a graph of immune responses in mice elicited by vaccinations with one of C-EL2, C-EL2r or KLH;

FIGS. 2A-2C are graphs of aggregation responses of platelets from mice vaccinated with one of C-EL2, C-EL2r or KLH;

FIGS. 3A-3C are graphs of dense granule secretion responses of platelets from mice vaccinated with one of C-EL2, C-EL2r or KLH;

FIG. 4A-4C are graphs of alpha (a) granule secretion responses of platelets from mice vaccinated with one of C-EL2, C-EL2r or KLH;

FIG. 5A-5D are graphs of integrin activation responses of platelets from mice vaccinated with one of C-EL2, C-EL2r or KLH;

FIGS. 6A-6B are graphs of occlusion times of thrombosis in mice vaccinated with one of C-EL2, C-EL2r or KLH;

FIG. 7A-7B are graphs of platelet-leukocyte aggregate formation responses in samples from mice vaccinated with one of C-EL2, C-EL2r or KLH;

FIG. 8 is a graph of the expression levels of major platelet integrins of platelets from mice with one of vaccinated with one of C-EL2, C-EL2r or KLH;

FIG. 9 is a graph of occlusion times of thrombosis in mice vaccinated with one of C-EL2, C-EL2r or KLH, and subsequently injected with the C-EL2 cognate peptide; and

FIG. 10 is a graph of aggregation responses of platelets from mice vaccinated with one of C-EL2, C-EL2r or KLH, and subsequently injected with the C-EL2 cognate peptide.

### DETAILED DESCRIPTION OF THE INVENTION

The illustrative embodiments recognize and take into account one or more different considerations. For example, the illustrative embodiments recognize and take into account that the thromboxane A₂/thromboxane A₂ receptor (TXA₂-TPR) signaling pathway is known to play an important role in platelet function in vivo, and has been implicated in the genesis of various forms of cardiovascular disorders. However, despite its clear involvement in such diseases, considerable gaps remain in our understanding of TPR's structural biology, which has hampered TPR-focused drug discovery.

Agents with TPR antagonistic activity would be expected to exhibit a better/safer pharmacological profile, and perhaps be more effective in managing thrombosis-based diseases. In contrast to the COX inhibitor aspirin, TS inhibitors exhibited minimal in vivo activity because the immediate precursor of TXA₂, PGH₂, binds to the same receptor, i.e., TPR and can therefore induce platelet aggregation.

A number of TPR antagonists were designed throughout the years and tested for biological activity. While in vitro results were encouraging, the in vivo effectiveness of these molecules was limited by short biological half-life, toxicity or limited tissue distribution.

One apparent reason for this failure is because these agents were empirically designed based on the complex structures of PGH₂ and/or TXA₂, with little information concerning the actual TPR binding domains. Resolution of these issues is crucial for the development of anti-thromboxane-based therapeutic strategies.

The illustrative embodiments recognize and take into account that, in spite of the clear involvement of TPR signaling in occlusive vascular disease, aspirin is still the only clinically effective drug for the prevention of TPR-mediated platelet activation. Thus, the availability of a pharmacologically effective non-aspirin derivative or C-EL2-derived vaccine with anti-TPR activity could have widespread therapeutic applications, especially given the limitations of current thromboembolic therapy, e.g., resistance, and bleeding associated with COX inhibitor aspirin.

The illustrative embodiments described herein constitute the first investigation of vaccine-based antithrombotic agent, and of immunization-based inhibition of TPR function in vivo. Moreover, the illustrative embodiments also provide novel information concerning a potential target site, i.e., C-EL2, for therapeutic intervention protecting against thrombosis. Finally, the functionally active TPR sequence identified herein significantly aid molecular modeling study predictions for organic derivatives which possess in vivo activity.

Based on these considerations, the illustrative embodiments provide a method and vaccine for treating thrombotic conditions that selectively targets the TXA₂/TPR pathway and blocking TXA₂-mediated platelet aggregation.

An example of the amino acid sequence of a TPR is provided in GenBank accession number BAA07274.1, which has the amino acid sequence:

SEQ ID NO: 1
MWPNGSSLGPCFRPTNITLEERRLIASPWFAASFCVVGLAS
NLLALSVLAGARQGGSHTRSSFLTFLCGLVLTDFLGLLVTGTIV
VSQHAALFEWHAVDPGCRLCRFMGVVMIFFGLSPLLLGAAMASE
RYLGITRPFSRPAVASQRRAWATVGLVWAAALALGLLPLLGVGR
YTVQYPGSWCFLTLGAESGDVAFGLLFSMLGGLSVGLSFLLNTV
SVATLCHVYHGQEAAQQRPRDSEVEMMAQLLGIMVVASVCWLPL
LVFIAQTVLRNPPAMSPAGQLSRTTEKELLIYLRVATWNQILDP
WVYILFRRAVLRRLQPRLSTRPRSLSLQPQLTQRSGLQ.

Research efforts to map the TPR ligand binding domain revealed that the TPR ligand binding domain resides in the C-terminus of the second extracellular loop (CEL2; C183-D193) of the receptor protein.

The C-EL2 segment of SEQ ID NO:1, C-EL2 includes the amino acid sequence:

SEQ ID NO: 2
CFLTLGAESGD

or variants thereof. This extracellular segment contains ligand-amino acid coordination sites.

An antibody raised against this sequence (i.e., C-EL2Ab) inhibits TPR ligand binding, platelet aggregation in vitro, and protects from thrombogenesis in vivo without any apparent bleeding diathesis or interference with physiological hemostasis, making it the first functional antibody against platelet TPRs.

The illustrative embodiments are directed to a therapeutic C-EL2 TPR vaccine. The effects of therapeutic C-EL2 TPR vaccine are predominantly limited to platelet TPRs, in part because the distribution of C-EL2 antibody to compartments other than the vascular system is, in general, restricted due to poor penetration of the endothelial cell layer. As a result, C-EL2 TPR vaccine approaches address bleeding time and thrombosis under conditions of selective modulation of the platelet TPRs, without affecting TPRs of the smooth muscle—which are known to affect bleeding time.

In an illustrative embodiment, a vaccine of the cognate TPR C-EL2 peptide is employed as an immunogen to in vivo TPR-dependent platelet activation, e.g., hemostasis/bleeding time, and the genesis of thrombosis. The CEL2 peptide TPR vaccine induced production of a C-EL2 TPR antibody, and inhibited aggregation induced by TPR agonists. Additionally, the CEL2 peptide TPR vaccine produced no effects on aggregation stimulated by separate agonists, such as ADP and TRAP4. Moreover, the CEL2 peptide TPR vaccine inhibited TPR-dependent dense and alpha granule release, and GPIIb-IIIa.

The TPR C-EL2-based vaccine of the illustrative embodiments protects against thrombogenesis, without impairing hemostasis. Amongst other advantages, this active vaccination approach would not be expected to face some of the functional limitations an antagonist does, including frequent administration and high costs.

The illustrative embodiments generally provide compositions that induce antibodies that bind thromboxane (TX) A₂ receptors (TPR) and inhibit thrombosis and other events within the cardiovascular, renal or pulmonary systems. Compositions of the invention prevent TXA₂ from binding to the TPR and stimulating platelet activation and aggregation, thereby decreasing the risk of a clinically significant thrombus or embolus. Thus, the C-EL2 vaccine provides beneficial pharmaceutical properties for treating thrombosis and other events within the cardiovascular, renal or pulmonary systems.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

### Vaccine Compositions

While vaccine development is a complex and challenging process, peptide-based vaccines (e.g., C-EL2) provide several advantages in comparison to conventional vaccines. Peptide vaccines are safer and more economic. Peptide vaccine production is also relatively inexpensive due to the ease of production and simplistic composition. Additionally, peptide vaccines avoid the inclusion of unnecessary components possessing high reactogenicity to the host, such as lipopolysaccharides, lipids, or toxins.

The present invention further provides compositions or vaccine compositions, comprising at least one active ingredient selected from (a) a peptide of the present invention; or (b) a polynucleotide encoding a peptide of the present invention.

The compositions of the present invention can comprise as needed a carrier(s), an excipient(s) or such commonly used in pharmaceuticals without particular limitations, in addition to the active ingredient(s) described above. An example of a carrier that can be used in a pharmaceutical composition of the present invention includes sterilized water, physiological saline, phosphate buffer, culture fluid and such. Therefore, the present invention also provides compositions, comprising at least one active ingredient and a pharmaceutically acceptable carrier: (a) a peptide of the present invention; or (b) a polynucleotide encoding a peptide of the present invention in an expressible form.

Further, the vaccine compositions of the present invention can comprise, as needed, stabilizers, suspensions, preservatives, surfactants, solubilizing agents, pH adjusters, aggregation inhibitors and such.

The compositions comprising an active agent as described herein can be used as a vaccine. In the context of the present invention, the term “vaccine” (also called “immunogenic composition”) refers to a composition that has a function of inducing an immune response that leads to TPR modulation when inoculated into an animal. Thus, a C-EL2 pharmaceutical composition can be used to induce an immune response that leads to therapeutic action. The immune response induced by a peptide or a polypeptide and a pharmaceutical composition of the present invention is not particularly limited as long as it is an immune response that leads to anti-TPR action.

In the present invention, peptides having the amino acid sequence of SEQ ID NO: 2 or a functional variant thereof are peptides that can induce a potent and specific immune response. Therefore, pharmaceutical compositions of the present invention comprising a peptide having the amino acid sequence of SEQ ID NO: 2 or a functional variant thereof is suitable for administration to a subject in need of modulation of TPR. The same applies to pharmaceutical compositions comprising a polynucleotide encoding such peptides.

The pharmaceutical compositions of the present invention may also optionally comprise the other therapeutic substances as an active ingredient, as long as they do not inhibit the anti-TPR effects of the above-described active ingredients such as peptides of the present invention.

It should be understood that in consideration of the formulation type, the pharmaceutical composition of the present invention may include other components conventional in the art, in addition to the ingredients specifically mentioned herein.

Embodiments can include articles of manufacture or kits that comprise a vaccine composition or components described herein. The articles of manufacture or kits of the present invention can include a container that houses a composition described herein. An example of an appropriate container includes a bottle, a vial or a test tube, but is not limited thereto. The container may be formed of various materials such as glass or plastic. A label may be attached to the container, and the disease or disease state to which the pharmaceutical composition of the present invention should be used may be described in the label. The label may also indicate directions for administration and such.

The articles of manufacture or kits of the present invention may further comprise a second container that houses pharmaceutically acceptable diluents optionally, in addition to the container that houses the pharmaceutical composition of the present invention. The articles of manufacture or kits of the present invention may further comprise the other materials desirable from a commercial standpoint and the user's perspective, such as the other buffers, diluents, filters, injection needles, syringes, package inserts with instructions for use.

As needed, the vaccine composition or components thereof can be provided in a pack or dispenser device that can contain one or more units of dosage forms containing active ingredients.

The vaccine composition comprising a peptide described herein or functional variant thereof can be formulated by conventional formulation methods as needed. The pharmaceutical compositions of the present invention may comprise as needed in addition to the active ingredient, carriers, excipients and such commonly used in pharmaceuticals without particular limitations. Examples of carriers that can be used in pharmaceutical compositions of the present invention include sterilized water (for example, water for injection), physiological saline, phosphate buffer, phosphate buffered saline, Tris buffered saline, 0.3% glycine, and such. Further, the pharmaceutical compositions of the present invention may comprise as needed stabilizers, suspensions, preservatives, surfactants, solubilizing agents, pH adjusters, aggregation inhibitors, and such. The pharmaceutical compositions of the present invention can induce specific immunity against TPR, and thus can be applied for the purpose of treatment or prevention (prophylaxis).

For example, the vaccine compositions can be prepared by dissolving in pharmaceutically or physiologically acceptable water-soluble carriers such as sterilized water (for example, water for injection), physiological saline, phosphate buffer, phosphate buffered saline, and Tris buffered saline and adding, as needed, stabilizers, suspensions, preservatives, surfactants, solubilizing agents, pH adjusters, aggregation inhibitors and such, and then sterilizing the peptide solution. The method of sterilizing a peptide solution is not particularly limited, and is preferably carried out by filtration sterilization. The filtration-sterilized peptide solution can be administered to a subject, for example, as an injection, without being limited thereto. The vaccine compositions may be prepared as a freeze-dried formulation by freeze drying the above-described peptide solution. The freeze-dried formulation can be prepared by filling the peptide solution into an appropriate container such as an ampule, a vial or a plastic container, followed by freeze drying and encapsulation into the container with a wash-sterilized rubber plug or such after pressure recovery. The freeze-dried formulation can be administered to a subject after it is re-dissolved in pharmaceutically or physiologically acceptable water-soluble carriers such as sterilized water (for example, water for injection), physiological saline, phosphate buffer, phosphate buffered saline, Tris buffered saline and such before administration. Preferred examples of compositions include injections of such a filtration-sterilized peptide solution, and freeze-dried formulations that result from freeze-drying the peptide solution. Certain embodiments encompass kits comprising such a freeze-dried formulation and redissolving solution. The present invention also encompasses kits comprising a container that houses the freeze-dried formulation, which is a pharmaceutical composition of the present invention, and a container that houses a re-dissolving solution thereof.

The vaccine compositions can comprise a combination of two or more types of the peptides of the present invention. The combination of peptides can take a cocktail form of mixed peptides, or can be conjugated with each other using standard techniques. For example, peptides can be chemically linked or expressed as single fusion polypeptide sequences. In certain aspects a second peptide is an adjuvant for enhancing the immunogenicity of the first peptide.

Examples of suitable adjuvants include peptides such as Keyhole Limpet hemocyanin protein (KLH); aluminum salts (aluminum phosphate, aluminum hydroxide, aluminum oxyhydroxide and such); alum; cholera toxin; Salmonella toxin; Incomplete Freund's adjuvant (IFA); Complete Freund's adjuvant (CFA); ISCOMatrix; GM-CSF and other immunostimulatory cytokines; oligodeoxynucleotide containing the CpG motif (CpG7909 and such); oil-in-water emulsions; Saponin or its derivatives (QS21 and such); lipopolysaccharide such as Lipid A or its derivatives (MPL, RC529, GLA, E6020 and such); lipopeptides; lactoferrin; flagellin; doublestranded RNA or its derivatives (poli IC and such); bacterial DNA; imidazoquinolines (Imiquimod, R848 and such); C-type lectin ligand (trehalose-6,6′-dibehenate (TDB) and such); CD1d ligand (alpha-galactosylceramide and such); squalene emulsions (MF59, AS03, AF03 and such); PLGA; virus-like particles; and the like, without being limited thereto. The adjuvant may be contained in the same or another container separate from the peptide composition in the kits comprising vaccine components. In this case, the adjuvant and the peptide composition may be administered to a subject in succession, or mixed together immediately before administration to a subject. Such kits comprising a vaccine composition comprising a peptide and an adjuvant are also provided. When the vaccine composition is a freeze-dried formulation, the kit can further comprise a redissolving solution. Further, embodiments include kits comprising a container that houses a peptide composition and a container that stores an adjuvant. The kit can further comprise as needed a container that stores the re-dissolving solution.

When an oil adjuvant is used as an adjuvant, the composition may be prepared as an emulsion. Emulsions can be prepared, for example, by mixing and stirring the peptide solution and an oil adjuvant. The peptide solution may be one that has been re-dissolved after freeze drying. The emulsion may be either of the W/O-type emulsion and O/W-type emulsion, and the W/O-type emulsion is preferred for obtaining a high immune response-enhancing effect. IFA can be preferably used as an oil adjuvant, without being limited thereto. Preparation of an emulsion can be carried out immediately before administration to a subject, and in this case, the vaccine composition may be provided as a kit comprising the peptide solution and an oil adjuvant. When the pharmaceutical composition is a freeze-dried formulation, the kit can further comprise a redissolving solution.

Further, the pharmaceutical composition of the present invention may be a liposome formulation within which a peptide is encapsulated, a granular formulation in which a peptide is bound to beads with several micrometers in diameter, or a formulation in which a lipid is bound to a peptide.

In another embodiment of the present invention, the peptide may also be administered in the form of a pharmaceutically acceptable salt. Preferred examples of salts include salts with alkali metals (lithium, potassium, sodium and such), salts with alkaline-earth metals (calcium, magnesium and such), salts with other metals (copper, iron, zinc, manganese and such), salts with organic bases, salts with amines, salts with organic acids (acetic acid, formic acid, propionic acid, fumaric acid, maleic acid, succinic acid, tartaric acid, citric acid, malic acid, oxalic acid, benzoic acid, methanesulfonic acid and such), and salts with inorganic acids (hydrochloric acid, phosphoric acid, hydrobromic acid, sulfuric acid, nitric acid and such). The phrase “pharmaceutically acceptable salt” used herein refers to a salt that retains the biological, physiological, pharmacological and pharmaceutical efficacy and property of the compound. Therefore, pharmaceutical compositions comprising a pharmaceutically acceptable salt of a peptide of the present invention are also encompassed by the present invention. Further, the “peptide of the present invention” also encompasses, in addition to the free peptide, pharmaceutically acceptable salts thereof.

Examples of suitable methods for administering the peptides or vaccine compositions include oral, epidermal, subcutaneous, intramuscular, intraosseous, peritoneal, intranasal, and intravenous injections, as well as systemic administration or local administration, but are not limited thereto. The administration can be performed by single administration or boosted by multiple administrations. The peptides can be administered to a subject in a therapeutically or pharmaceutically effective amount. The dose of the peptides of the present invention can be appropriately adjusted according to the disease to be treated, the patient's age and weight, the method of administration and such. For each of the peptides of the present invention, the dose is usually 0.001 mg-1000 mg, for example, 0.01 mg-100 mg, for example, 0.1 mg-30 mg, for example, 0.1 mg-10 mg, for example, 0.5 mg-5 mg. The dosing interval can be once every several days to several months, and for example, the dosing can be done in a once-per-week interval. A skilled artisan can appropriately select a suitable dose (dosage).

The vaccine compositions include a therapeutically effective amount of a peptide, a pharmaceutically or physiologically acceptable carrier, and an adjuvant. The pharmaceutical compositions of the present invention can comprise 0.001 mg-1000 mg, preferably 0.01 mg-100 mg, more preferably 0.1 mg-30 mg, even more preferably 0.1 mg-10 mg, for example, 0.5 mg-5 mg of a peptide. When a vaccine composition is an injection, it can comprise a peptide at a concentration of 0.001 mg/ml-1000 mg/ml, preferably 0.01 mg/ml-100 mg/ml, more preferably 0.1 mg/ml-30 mg/ml, even more preferably 0.1 mg/ml-10 mg/ml, for example, 0.5 mg/ml-5 mg/ml. In this case, for example, 0.1 to 5 ml, preferably 0.5 ml to 2 ml of the pharmaceutical composition of the present invention can be administered to a subject by injection.

As used herein, the term “variant” (or analog) polynucleotide or peptide refers to a polynucleotide or peptide differing from a specifically recited polynucleotide or peptide of the invention by insertions, deletions, and substitutions, created using, e.g., recombinant DNA techniques. Specifically, recombinant variants encoding these same or similar peptides may be synthesized or selected by making use of the “redundancy” in the genetic code. Various codon substitutions, such as the silent changes that produce various restriction sites, may be introduced to optimize cloning into a plasmid or viral vector or expression in a particular prokaryotic or eukaryotic system. Mutations in the polynucleotide sequence may be reflected in the peptide or domains of other peptides added to the peptide to modify the properties of any part of the peptide, to change characteristics such as ligand-binding affinities, interchain affinities, or degradation/turnover rate.

As used herein, the term “variant C-EL2 peptide” refers to a molecule that differs in amino acid sequence from a “parent” C-EL2 peptide amino acid sequence (e.g., SEQ ID NO:2) by virtue of addition, deletion and/or substitution of one or more amino acid residue(s) in the parent antibody sequence. For example, the variant may comprise at least one to about three substitutions. A variant C-EL2 peptide may also comprise one or more additions, deletions and/or substitutions in one or more amino acids. The variant peptide will retain the ability to induce an immune response to TPR C-EL2.

Amino acid “substitutions” can result in replacing one amino acid with another amino acid having similar structural and/or chemical properties, e.g., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are generally in the range of about 1 to about 20 amino acids, more specifically about 1 to about 10 amino acids, and even more specifically, about 2 to about 5 amino acids. Non-conservative substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). The variation allowed may be experimentally determined by systematically making insertions, deletions, or substitutions of amino acids in a peptide molecule using recombinant DNA techniques and assaying the resulting recombinant variants for activity.

Certain embodiments are directed to an expression vector and/or a host cell that comprises one or more polynucleotide sequence encoding a peptide described herein. For example, the host cell or expression vector comprises any one or more of the polynucleotides or polynucleotides encoding the peptides and/or functional variants thereof. In a specific embodiment, the host cell or expression vector comprises one or more polynucleotides encoding peptide(s) provided herein.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The illustrative examples use a mouse keyhole limpet hemocyanin/peptide-based vaccination approach rationalized over the TPR ligand-binding domain, i.e., the C-terminus of the second extracellular loop. Of note, the mouse and the human platelet TPRs are identical in 17 out of the 21 amino acid that are located in the second extracellular loop region of the receptor protein. The biological activity of this vaccine was assessed in the context of platelets and thrombotic diseases, and using a host of in vitro and in vivo platelet function experiments.

Mice were immunized with keyhole limpet hemocyanin-(KLH) coupled peptide corresponding to SEQ ID NO. 2. Control animals are immunized with a randomized peptide having the amino acid sequence

SEQ ID NO. 3:
STLACGFDGEL

An additional cysteine synthesized at the end of the peptides allow coupling with KLH (CSTLACGFDGEL), or with KLH alone. Mice received intraperitoneal injections of peptides (35 μg) or KLH dissolved in Freund's complete adjuvant. The animals were boosted 3 times with peptides (and Freund's incomplete adjuvant).

Mouse blood was collected from a ventricle and the citrated (0.38%) blood was mixed with phosphate-buffered saline, pH 7.4, and was incubated with PGI₂ (10 ng/mL; 5 minutes), followed by centrifugation at 237×g for 10 minutes at room temperature (RT). Platelet-rich plasma (PRP) was recovered and platelets were pelleted at 483×g for 10 minutes at RT. The pellets were resuspended in HEPES/Tyrode buffer (HT; 20 mM HEPES/KOH, pH 6.5, 128 mM NaCl, 2.8 mM KCl, 1 mM MgCl₂, 0.4 mM NaH₂PO₄, 12 mM NaHCO₃, 5 mM d-glucose) supplemented with 1 mM EGTA, 0.37 U/mL apyrase, and 10 ng/mL PGI₂. Platelets were washed and resuspended in HT (pH 7.4) without EGTA, apyrase, or PGI₂. Platelets were counted with an automated hematology analyzer (Drew Scientific Dallas, Tex.) and adjusted to the indicated concentrations.

All experiments were performed at least three times. Analysis of the data was performed using GraphPad PRISM statistical software (San Diego, Calif.) by employing the t-test, and data presented as mean±SEM, or mean±SD, as specified. The Mann Whitney test was used for the evaluation of differences in mean occlusion and bleeding times. Analysis was also conducted using t-test, and similar results were obtained. Significance was accepted at P<0.05 (two-tailed P value), unless stated otherwise.

All experiments involving animals were performed in compliance with the institutional guidelines and were approved by the Institutional Animal Care and Use Committee.

### Results

The results revealed that the TPR C-terminus of the second extracellular loop vaccine: (1) triggered an immune response, which resulted in the development of a C-terminus of the second extracellular loop antibody; (2) did not affect expression of major platelet integrins (e.g., glycoprotein IIb-IIIa); (3) selectively inhibited TPR-mediated platelet aggregation, platelet-leukocyte aggregation, integrin glycoprotein IIb-IIIa activation, as well as dense and a granule release; (4) significantly prolonged thrombus formation; and (5) did so without impairing physiological hemostasis.

1. Antibody Production.

Enzyme-linked immunosorbent assays (ELISAs) were performed to determine antibody development in the immunized mice (C-EL2 Vac, CEL2r Vac, and KLH). Nunc-Immuno™ MicroWell™ 96-Well plates were coated with 12.5 μg/well C-EL2 peptide for 18-24 h at room temperature. Following the incubation, the plates were washed three times with 200 μL/well modified Tyrode's buffer (0.1% bovine serum albumin, 20 mM HEPES/KOH, pH 7.4, 128 mM NaCl, 2.8 mM KCl, 1 mM MgCl₂, 0.4 mM NaH₂PO₄, 12 mM NaHCO₃, 5 mM d-glucose), and then nonspecific sites were blocked by incubation for 1 h with 5% bovine serum albumin (200 μL/well) in the same buffer. Plates were again washed three times with the modified Tyrode's buffer prior to applying serial dilutions of C-EL2 peptide to the wells in triplicate. Antibodies were allowed to incubate for 1 h followed by three more washes. Antibodies bound to the immobilized peptide were detected by incubation (1 h) with goat anti-mouse IgG (heavy+light) conjugated to horseradish peroxidase. The wells were then washed a final time before the addition of the horseradish peroxidase substrate solution. After 10-min incubation in the dark, the reaction was quenched with 2 N H₂SO₄ (200 μL/well). The presence of specific antibodies, i.e., the C-EL2 antibody/immunoglobulin G, was measured by the absorbance at 490 nm.

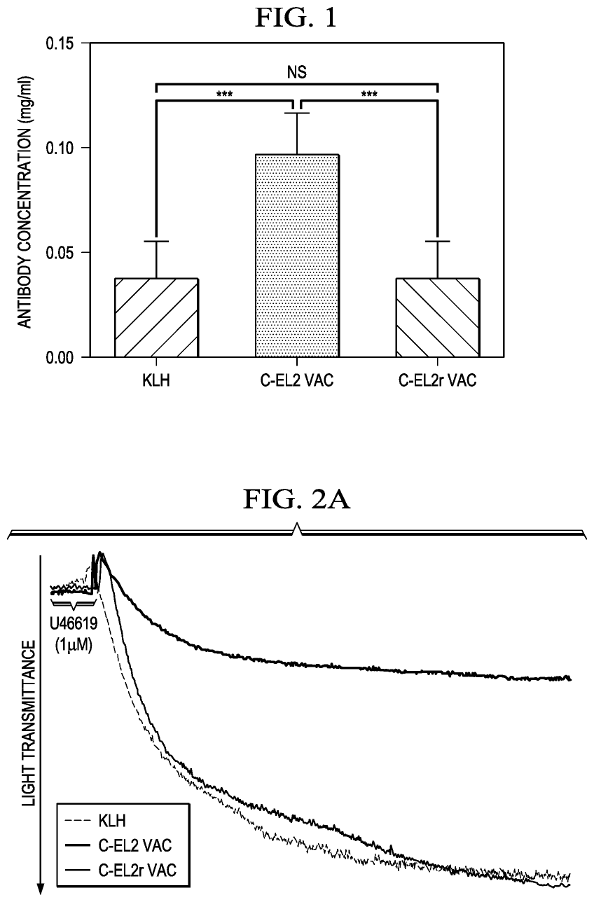

As indicated in FIG. 1, significant levels of the C-EL2 antibody was observed when C-EL2 was used as an immunogen, unlike C-EL2r and KLH. The relative antibody concentrations illustrated in indicates that C-EL2 vaccination successfully elicits an immune response targeting the C-terminus of the second extracellular loop of the thromboxane A₂ receptor.

After immunization, peripheral blood samples were analyzed to determine whether the C-EL2 vaccine would modulate platelet and other peripheral blood cell counts. Peripheral Blood Cell Counts in KLH, C-EL2, and C-EL2r Vaccinated Mice are indicated in Table 1.

TABLE 1

| | KLH | C-EL2 Vac | C-EL2r Vac | P values* |
|---|---|---|---|---|
| Platelets | 1087.48 ± 41.2 | 1154.13 ± 38.7 | 1121 ± 55.4 | NS |
| MPV | 4.63 ± 2.44 | 4.81 ± 2.78 | 4.94 ± 3.12 | NS |
| Red Blood Cells | 8.13 ± 1.39 | 8.76 ± 1.44 | 8.35 ± 1.21 | NS |
| Lymphocytes | 6.33 ± 1.72 | 6.91 ± 1.38 | 6.65 ± 1.41 | NS |
| Monocytes | 0.039 ± 0.024 | 0.042 ± 0.031 | 0.044 ± 0.021 | NS |
| Granulocytes | 2.51 ± 2.22 | 2.36 ± 2.61 | 2.43 ± 2.09 | NS |

All counts are thousands per microliter, except for red blood cells, which are millions per microliter. *NS: not significant; comparisons were made between KLH and C-EL2 Vac, as well as C-EL2 Vac and C-EL2r Vac. Data are represented as mean±SD.

Vaccinations with the C-EL2 antigen show a normal platelet count, and other blood parameters, relative to the control. As indicated, the C-EL2 TPR vaccine elicits an immune response, without altering peripheral blood count.

Platelet count were measured 1, 2, and 3 months after the final boost of C-EL2 vaccine. Time Course of Platelet Count and Antibody Titer in C-EL2 Vaccinated Mice are indicated in Table 2.

TABLE 2

| | Antibody Titer, | | |
|---|---|---|---|
| Time, mo | mg/ml | Platelet Count | P values* |
| 1 | 0.91 ± 0.018 | 1102.26 ± 29.8 | NS |
| 2 | 0.84 ± 0.022 | 1135.18 ± 41.1 | NS |
| 3 | 0.79 ± 0.027 | 1116.73 ± 48.9 | NS |

No differences in the measured platelet counts over the time course. However, the antibody titers remained relatively “high.” These data suggest that vaccinations with the C-EL2 antigen do not affect the “hematology” profile.

2. Platelet Aggregation

Aggregation response was measured to determine whether in vivo immunizations with the C-EL2 peptide treatment would also translate into inhibition of platelet aggregation by the C-EL2 vaccine (i.e., antibody generated from this vaccine).

Blood was collected from mice that were vaccinated with C-EL2, CEL2r, and KLH, and the platelets were harvested by centrifugation. Platelets were stimulated with 1 μM U46619 (A), 5 μM ADP (B), or 80 μM thrombin receptor-activating peptide 4 (TRAP4; C). Aggregation response was monitored using an aggregometer.

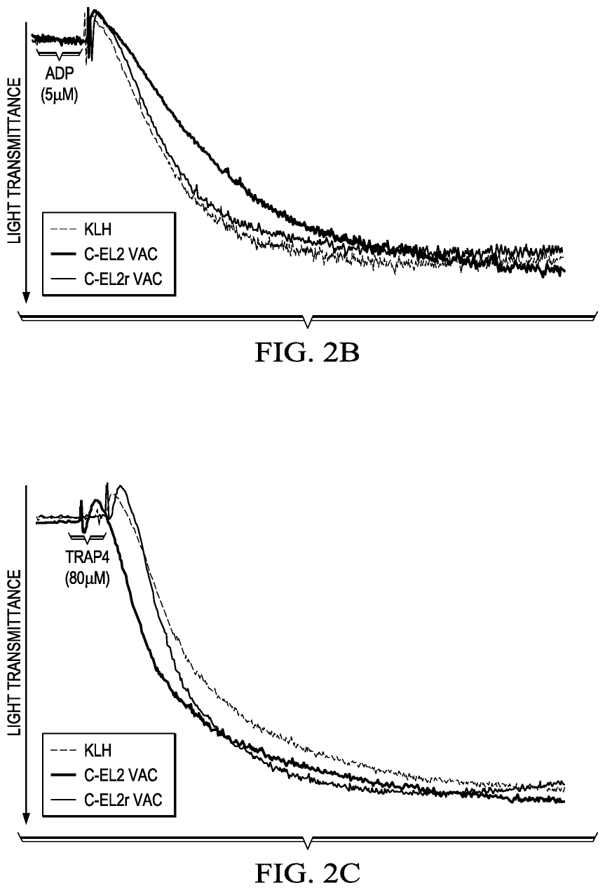

As indicated in FIGS. 2A-2C, vaccination with C-EL2 (35 μg), unlike C-EL2r or KLH, resulted in inhibition of 1 μM U46619-stimulated platelet aggregation (FIG. 2A). However, vaccination with C-EL2 (35 μg) had no effects on that induced by either ADP (5 μM; FIG. 2B) or TRAP4 (80 μM; FIG. 2C). These data suggest that C-EL2 TPR vaccine has the capacity to selectively inhibit platelet aggregation.

3. ATP & Alpha Granule Release

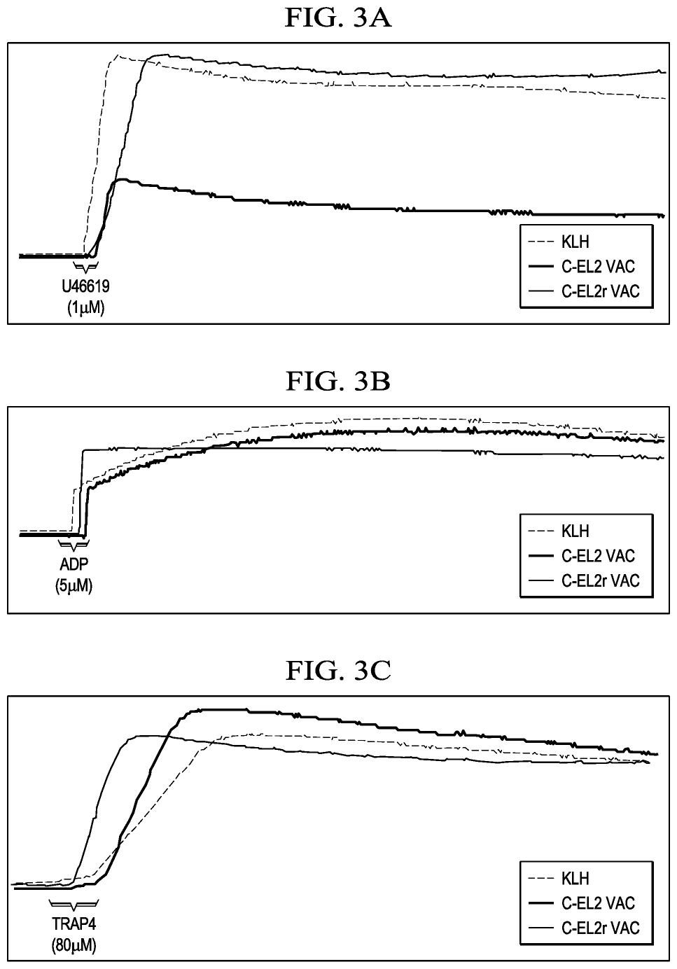

Platelet secretion is an important and early event in platelet activation, and is known to be triggered by TXA₂. As illustrated in FIGS. 3A-3C, C-EL2 TPR vaccine inhibits platelet dense granule and alpha granule secretion.

Platelets from vaccinated mice (C-EL2, C-EL2r and KLH) were prepared as described above (250 μL; 2.5×10⁸/mL) before being placed into siliconized cuvettes and stirred for 5 min at 37° C. at 1200 rpm. The luciferases substrate/luciferase mixture (12.5 μL, Chrono-Log) was then added, followed by the addition of the agonists U46619 (1 μM), ADP (5 μM) or TRAP4 (80 μM). ATP release (for dense granules) was detected as luminescence, and measured by a lumiaggregometer.

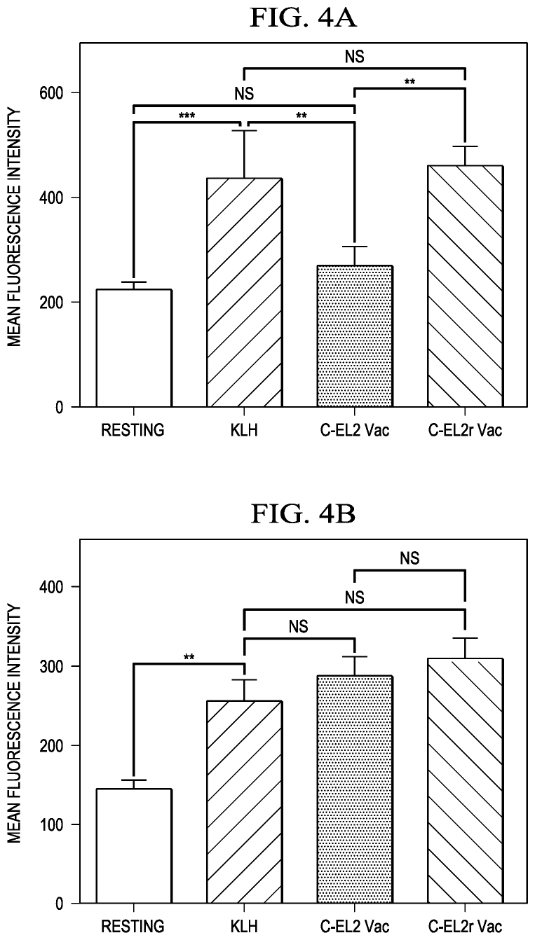

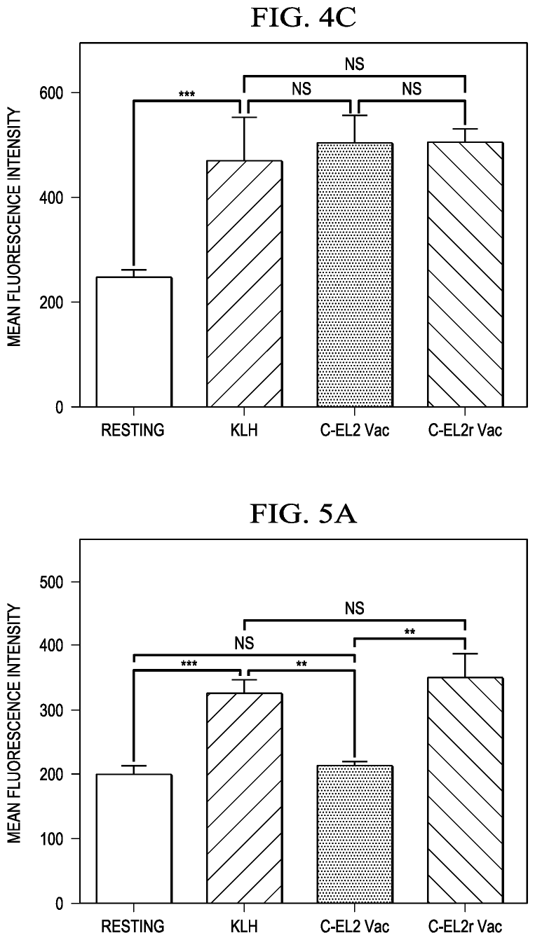

The C-EL2 (35 μg) vaccine inhibited platelet dense granule secretion (ATP release), and alpha granule secretion (P-selectin expression), in response to the TPR agonist U46619 (1 μM; FIGS. 3A and 4A), when compared with C-EL2r vaccine or KLH. However, the C-EL2 vaccine did not inhibit ATP release and P-selectin expression, in response to ADP (5 μM; FIGS. 3B and 4B) or TRAP4 (80 μM; FIGS. 3C and 4C), when compared with C-EL2r vaccine or KLH. These data show that that C-EL2 TPR vaccine exerts inhibitory effects on platelet granule secretion.

4. Integrin GPIIb-IIIa Activation and P-Selectin Expression.

It is well documented that integrin glycoprotein IIb-IIIa (aIIbb3) plays a crucial role in platelet aggregation in response to physiological agonists, and that it mediates thrombus formation. Having established that the TPR C-EL2 vaccine has the capacity to inhibit platelet aggregation, it was determined whether the TPR C-EL2 vaccine would be associated with a commensurate inhibition of integrin aIIbb3 activation.

Flow cytometric analysis was carried out on platelets from vaccinated mice (C-EL2, C-EL2r and KLH) as discussed before. Briefly, platelets (2×10⁸) were stimulated 1 μM U46619, 5 μM ADP, or 80 μM TRAP4 for 3 minutes. The reactions were stopped by fixing the platelets with 2% formaldehyde for 30 min at room temperature. Finally, platelets were incubated with FITC-conjugated JON/A or anti-P-selectin antibodies at room temperature for 30 min in the dark. Finally, the platelets were diluted 2.5-fold with HEPES/Tyrode buffer (pH 7.4). The samples were transferred to FACS tubes and fluorescent intensities were measured using a BD Accuri C6 flow cytometer and analyzed using CFlow Plus (BD Biosciences, Franklin Lakes, N.J.).

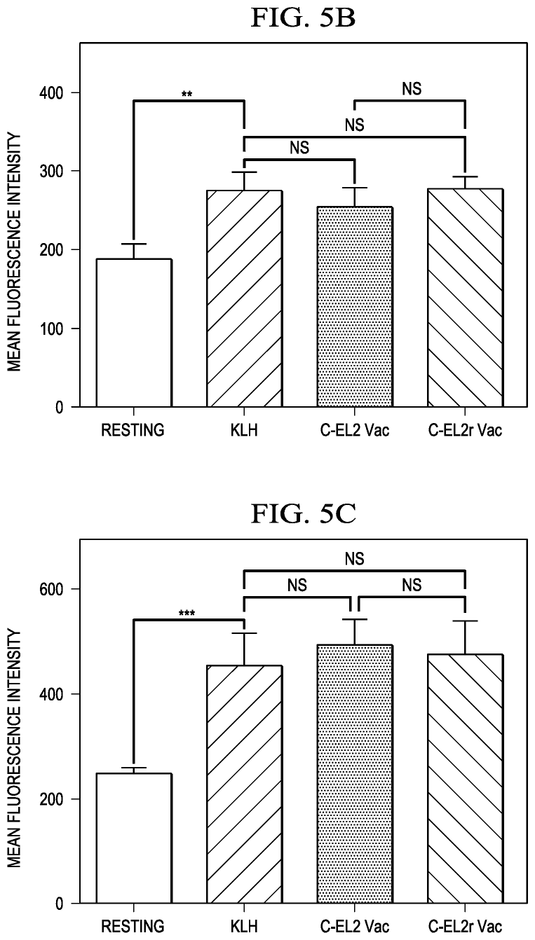

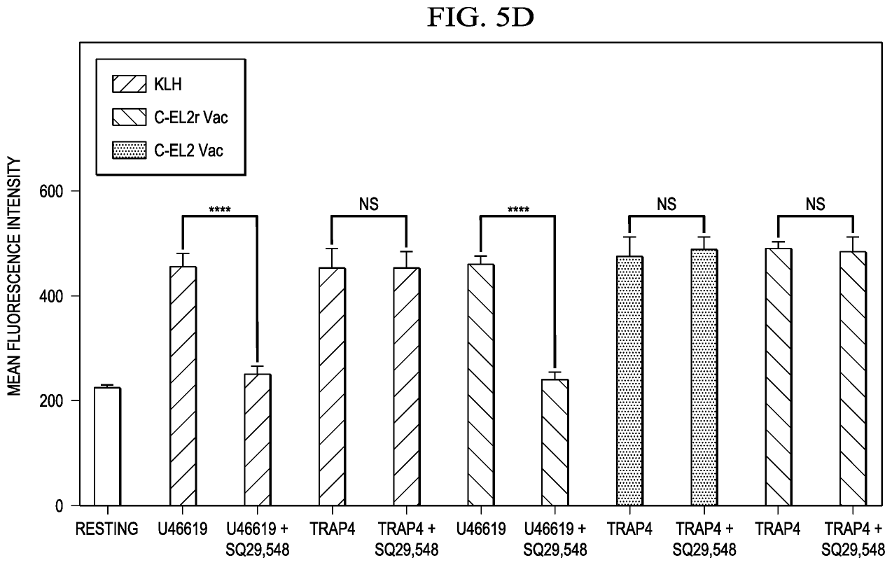

The C-terminus of the second extracellular loop vaccine (C-EL2 Vac), but not the random C-EL2 (C-EL2r) or keyhole limpet hemocyanin (KLH), inhibits integrin activation. Immunization with C-EL2 antigen (35 μg), unlike C-EL2r or KLH, results in significant inhibition of U46619-triggered JON/A binding (1 μM; FIG. 5A), but not that induced by ADP (5 μM; FIG. 5B) or TRAP4 (80 μM; 5C), which indicates abrogation of aIIbb3 activation. Moreover, the TPR antagonist SQ29,548 inhibited U46619 (1 μM)-mediated glycoprotein IIb-IIIa activation in the KLH and C-EL2r vaccinations, but had no effect on TRAP4 (80 μM)-triggered integrin activation, in any of the immunizations (FIG. 5D).

5. Occlusion & Bleeding Times

The C-terminus of the second extracellular loop vaccine (C-EL2 Vac) prolongs the time for occlusion, but not the tail bleeding time, whereas the random C-EL2 (C-EL2r) or keyhole limpet hemocyanin (KLH) has no effect.

Briefly, vaccinated mice (C-EL2, C-EL2r and KLH) were anesthetized with isoflurane. Then, the left carotid artery was exposed and cleaned, and baseline carotid artery blood flow was measured with Transonic micro-flowprobe (0.5 mm, Transonic Systems Inc., Ithaca, N.Y.). After stabilization of blood flow, 7.5% ferric chloride (FeCl₃) was applied to a filter paper disc (1-mm diameter) that was immediately placed on top of the artery for 3 min. Blood flow was continuously monitored for 45 min, or until blood flow reached stable occlusion (zero blood flow for 2 min). Data was recorded and time to vessel occlusion was calculated as the difference in time between stable occlusion and removal of the filter paper (with FeCl₃). An occlusion time of 45 min was considered as the cut-off time for the purpose of statistical analysis.

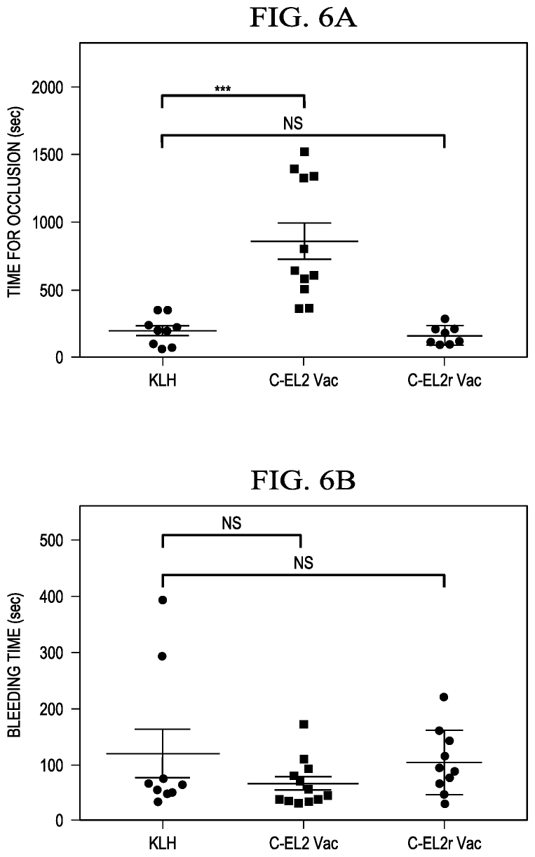

Mice that were immunized with C-EL2 peptide (35 μg), and subjected to the FeCl₃ carotid artery thrombosis model, exhibited a prolonged time for arterial thrombosis (FIG. 6A), relative to the controls CEL2r and KLH. Complete occlusion occurred by 2.5 minutes in the control animals, compared to more than 12 minutes in the C-EL2 vaccinated animals.

Hemostasis in the vaccinated mice (C-EL2, C-EL2r and KLH) was examined using the tail transaction technique. Briefly, mice were anesthetized with isoflurane and place on a 37° C. homeothermic blanket and their tails were transected 5 mm from the tip. The tail was placed in saline at 37° C. and the time to blood flow cessation was measured. Clotting was not considered complete until bleeding had stopped for 1 minute. When required, measurements were terminated at 15 minutes.

C-EL2 vaccinated mice had tail bleeding time that was no different from those that were vaccinated with C-EL2r or KLH (FIG. 6B). These results provide evidence that a C-EL2 TPR vaccine exerts anti-thrombotic activity, without increasing the risk of bleeding.

6. Platelet-Leukocyte Aggregate Formation

Platelet-leukocyte aggregates are known to contribute to the pathogenesis of thrombotic disorders, and TPR antagonists were found to reduce their formation. The C-terminus of the second extracellular loop vaccine (C-EL2 Vac) inhibits thromboxane A₂ receptor-induced, but not thrombin receptor-activating peptide 4 (TRAP4)-induced, platelet-leukocyte aggregate formation.

Blood from vaccinated mice (i.e., C-EL2, random C-EL2 [C-EL2r], or keyhole limpet hemocyanin [KLH]) was incubated with anti-P-selectin (platelet marker) and anti-CD11b (leukocyte marker) antibodies, before incubation with or without U46619 (1 μM). Events double positive for CD11b and P-selectin identified platelet-leukocyte aggregates and were recorded as a percentage of a total of 10000 gated leukocytes (KLH vs KLH+U46619, **P<0.001; CEL2 Vac vs C-EL2 Vac+U46619, P=not significant [NS]; C-EL2r Vac vs C-EL2r Vac+U46619, **P<0.001).

Blood from vaccinated mice (i.e., C-EL2, C-EL2r, or KLH) was incubated with anti-P-selectin (platelet marker) and anti-CD11b (leukocyte marker) antibodies, before incubation with or without TRAP4 (80 μM). Events double positive for CD11b and P-selectin identified platelet-leukocyte aggregates and were recorded as a percentage of a total of 10000 gated leukocytes (KLH vs KLH+TRAP4, *P<0.05; C-EL2 Vac vs C-EL2 Vac+TRAP4, **P<0.001; C-EL2r Vac vs C-EL2r Vac+TRAP4, **P<0.001). The error bars in this figure represent SEM. Each experiment was repeated 3 times using 3 separate groups, with blood pooled from 8 mice per group.

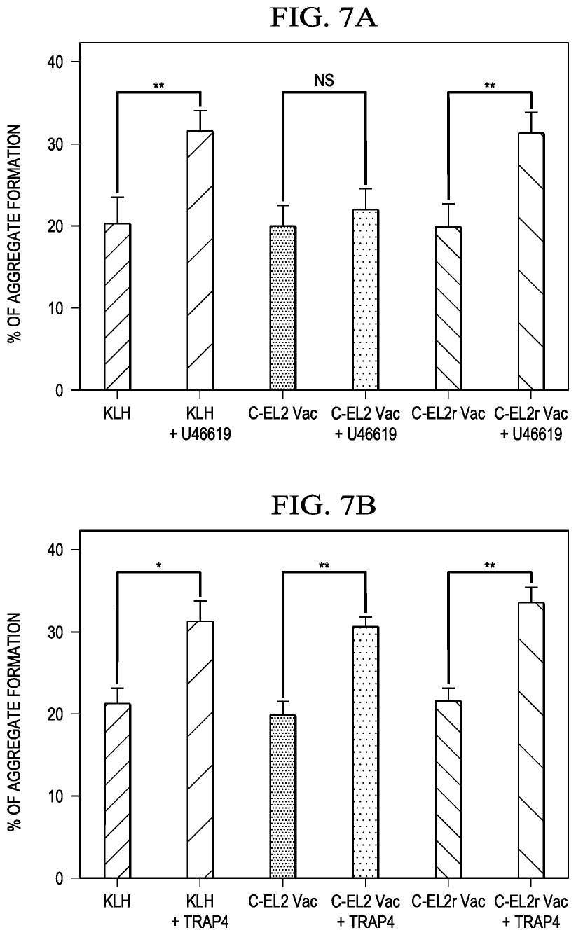

U46619 (1 μM)-induced platelet-leukocyte complexes are reduced with the CEL2 vaccine, but not with the KLH or the C-EL2r controls (FIG. 7A). On the other hand, neither the C-EL2 vaccine nor the controls exert any effects on TRAP4 (80 μM)-triggered platelet-leukocyte aggregation (FIG. 7B).

7. Platelet Glycoprotein IIb-IIIc, Ib, and VI Surface Expression

To exclude the possibility that the phenotype observed with the C-EL2 vaccine may derive, in part, from unintended effects on major platelet integrin receptors, their expression levels were measured. The C-terminus of the second extracellular loop vaccine (C-EL2 Vac) does not affect the expression levels of major platelet integrins.

Platelets from nonvaccinated/native and from vaccinated mice (i.e., C-EL2, random C-EL2 [C-EL2r], or keyhole limpet hemocyanin [KLH]) were washed and incubated with antibodies against glycoprotein IIb-IIIa (GPIIb-IIIa), GPIb, and GPVI (P=not significant [NS]), and the fluorescent intensities were measured by flow cytometry. The error bars in this figure represent SEM. Each experiment was repeated 3 times using 3 separate groups, with blood pooled from 6 mice per group.

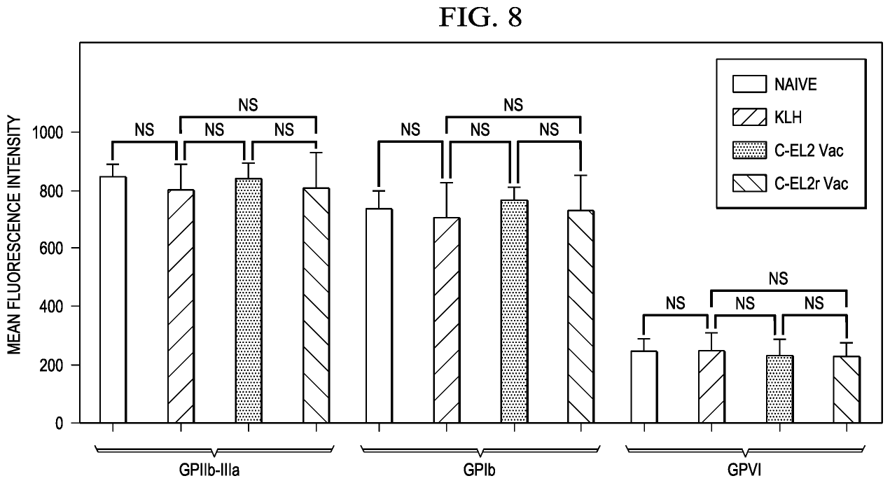

As can be seen in FIG. 8, immunizations with C-EL2, KLH, or C-EL2r produced no effects on the surface expression of glycoproteins IIb-IIIa, Ib, and VI. These findings further support the “specificity” of the effects produced by the C-EL2 vaccine.

8. Reversal of Vaccine Prolonged Occlusion Time

The C-EL2 vaccinated mice were injected with 8 mg/kg of the C-EL2 cognate peptide, and one-hour post injection were subjected, along with the KLH vaccinated mice to the FeCl₃ induced thrombosis model, as described above. The time for occlusion was then measured (C-EL2; NS, Mann-Whitney test). Each point represents the occlusion time of a single animal (C-EL2, n=4; and KLH, n=4).

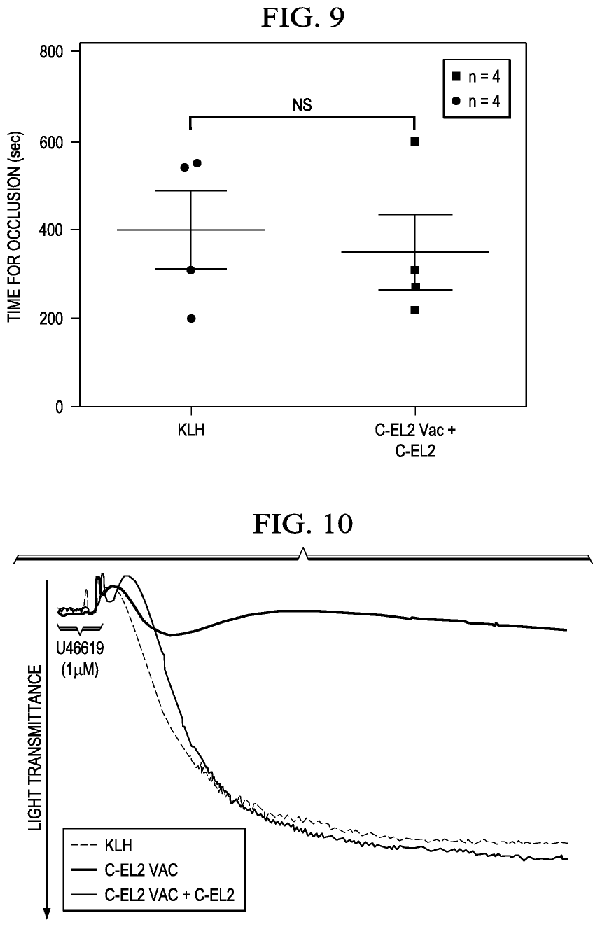

The C-EL2 vaccine prolongation of the time for occlusion is reversed by administering the C-EL2 cognate peptide (FIG. 9).

9. Reversal of Aggregation Inhibition

The C-EL2 vaccinated mice were first injected with 8 mg/kg of the C-EL2 cognate peptide, and one-hour post injection their platelets, along with those from non-cognate peptide injected C-EL2 as well as KLH vaccinated mice were collected. Platelets were then stimulated with 1 μM U46619 and their aggregation response was monitored using an aggregometer. Each experiment was repeated 3 times, with blood pooled from at least 6-8 mice each time.

The C-EL2 vaccine-mediated inhibition of aggregation is reversed by administering the C-EL2 cognate peptide (FIG. 10).

What is claimed is:

1. A vaccine for inducing an antibody response that binds to a thromboxane A₂ receptor (TPR), the vaccine comprising a C-EL2 peptide.

2. The vaccine of claim 1, wherein the C-EL2 peptide has an amino acid sequence of CFLTLGAESGD, or a functional variant thereof.

3. The vaccine of claim 2, wherein the functional variant is a variant C-EL2 peptide having one or more amino acid additions, deletions, substitution, or combinations thereof in the amino acid sequence of CFLTLGAESGD, wherein the functional variant retains the ability to induce the antibody response.

4. The vaccine of claim 1, wherein the C-EL2 peptide induces an antibody that specifically binds to the TPR and does not specifically bind to receptors of non-thromboxane agonists with different inhibitory aggregation pathways.

5. The vaccine of claim 1, wherein vaccine comprises an amount of C-EL2 peptide between 0.001 mg-1000 mg, preferably between 0.01 mg-100 mg, more preferably between 0.1 mg-30 mg, more preferably between 0.1 mg-10 mg, and specifically between 0.5 mg-5 mg of the C-EL2 peptide.

6. The vaccine of claim 1, wherein vaccine further comprises at least one additive selected from the group consisting of carriers, stabilizers, suspensions, preservatives, surfactants, solubilizing agents, pH adjusters, aggregation inhibitors, and combinations thereof.

7. The vaccine of claim 1, further comprising:
an adjuvant for enhancing the immunogenicity of the C-EL2 peptide, wherein the adjuvant is selected from a group consisting of peptides, aluminum salts, alum, bacterial toxins, Freund's adjuvants, immunostimulatory cytokines, oligodeoxynucleotides, oil-in-water emulsions, Saponin, lipopolysaccharides, lipopeptides, lactoferrin, flagellin, doublestranded RNA, bacterial DNA, imidazoquinolines, C-type lectin ligand, CD1d ligand, squalene emulsions, PLGA, and combinations thereof.

8. The vaccine of claim 1, wherein the vaccine is suitable for an administration method selected from the group consisting of oral, epidermal, subcutaneous, intramuscular, intraosseous, peritoneal, and intravenous injections.

9. The vaccine of claim 1, wherein the vaccine is administered as a pharmaceutically acceptable salt selected from the group consisting of salts of alkali metals, salts of alkaline-earth metals, salts of other metals, salts of organic bases, salts of amines, salts of organic acids, salts of inorganic acids, and combinations thereof.

10. A vaccine for inducing an antibody response that binds to a thromboxane A₂ receptor (TPR), the vaccine comprising a polynucleotide encoding a C-EL2 peptide.

11. The vaccine of claim 10, wherein the C-EL2 peptide has an amino acid sequence of CFLTLGAESGD, or a functional variant thereof.

12. The vaccine of claim 11, wherein the functional variant is a variant C-EL2 peptide having one or more amino acid additions, deletions, substitution, or combinations thereof in the amino acid sequence of CFLTLGAESGD, wherein the functional variant retains the ability to induce the antibody response.

13. The vaccine of claim 10, wherein the C-EL2 peptide induces an antibody that specifically binds to the TPR and does not specifically bind to receptors of non-thromboxane agonists with different inhibitory aggregation pathways.

14. The vaccine of claim 10, wherein vaccine comprises an amount of C-EL2 peptide between 0.001 mg-1000 mg, preferably between 0.01 mg-100 mg, more preferably between 0.1 mg-30 mg, more preferably between 0.1 mg-10 mg, and specifically between 0.5 mg-5 mg of the C-EL2 peptide.

15. The vaccine of claim 10, wherein vaccine further comprises at least one additive selected from the group consisting of carriers, stabilizers, suspensions, preservatives, surfactants, solubilizing agents, pH adjusters, aggregation inhibitors, and combinations thereof.

16. The vaccine of claim 10, further comprising:
an adjuvant for enhancing the immunogenicity of the C-EL2 peptide, wherein the adjuvant is selected from a group consisting of peptides, aluminum salts, alum, bacterial toxins, Freund's adjuvants, immunostimulatory cytokines, oligodeoxynucleotides, oil-in-water emulsions, Saponin, lipopolysaccharides, lipopeptides, lactoferrin, flagellin, doublestranded RNA, bacterial DNA, imidazoquinolines, C-type lectin ligand, CD1d ligand, squalene emulsions, PLGA, virus-like particles, and combinations thereof.

17. The vaccine of claim 10, wherein the vaccine is suitable for an administration method selected from the group consisting of oral, epidermal, subcutaneous, intramuscular, intraosseous, peritoneal, and intravenous injections.

18. A method of treating a condition comprising:
administering a pharmaceutically effective amount of a C-EL2 peptide, wherein the C-EL2 peptide induces an antibody response that binds to a thromboxane A₂ receptor (TPR).

19. The method of claim 18, wherein the C-EL2 peptide has an amino acid sequence of CFLTLGAESGD, or a functional variant thereof.

20. The method of claim 19, wherein the functional variant is a variant C-EL2 peptide having one or more amino acid additions, deletions, substitution, or combinations thereof in the amino acid sequence of CFLTLGAESGD, wherein the functional variant retains the ability to induce the antibody response.

21. The method of claim 18, wherein the C-EL2 peptide induces an antibody that specifically binds to the TPR and does not specifically bind to receptors of non-thromboxane agonists with different inhibitory aggregation pathways.

22. The method of claim 16, wherein pharmaceutically effective amount of the C-EL2 peptide is between 0.001 mg-1000 mg, preferably between 0.01 mg-100 mg, more preferably between 0.1 mg-30 mg, more preferably between 0.1 mg-10 mg, and specifically between 0.5 mg-5 mg of the C-EL2 peptide.


SEQUENCE LISTING (3 sequences)

SEQ ID NO: 1 - protein, 343 aa, unknown
  thromboxane A2 receptor
MWPNGSSLGPCFRPTNITLEERRLIASPWFAASFCVVGLASNLLALSVLAGARQGGSHTRSSFLTFLCGLVLTDFLGLLVTGTIVVSQHAALFEWHAVDPGCRLCRFMGVVMIFFGLSPLLLGAAMASERYLGITRPFSRPAVASQRRAWATVGLVWAAALALGLLPLLGVGRYTVQYPGSWCFLTLGAESGDVAFGLLFSMLGGLSVGLSFLLNTVSVATLCHVYHGQEAAQQRPRDSEVEMMAQLLGIMVVASVCWLPLLVFIAQTVLRNPPAMSPAGQLSRTTEKELLIYLRVATWNQILDPWVYILFRRAVLRRLQPRLSTRPRSLSLQPQLTQRSGLQ

SEQ ID NO: 2 - protein, 11 aa, Unknown
  C-terminus of the second extracellular loop of thromboxane A2 receptor
CFLTLGAESGD

SEQ ID NO: 3 - protein, 11 aa, Artificial Sequence
  Randomized peptide coupled to keyhole limpet hemocyanin- (KLH)
STLACGFDGEL